(19) United States
(12) Patent Application Publication
(10) Pub. No.: US 2022/0024943 A1
(43) Pub. Date: Jan. 27, 2022

(54) HIGH PURITY CANTHARIDIN MANUFACTURING PROCESS AND COMPOSITIONS

(71) Applicant: Sol-Gel Technologies Ltd., Ness Ziona (IL)

(72) Inventor: Stephen CHERKEZ, Caesarea (IL)

(73) Assignee: Sol-Gel Technologies Ltd., Ness Ziona (IL)

(21) Appl. No.: 17/384,820

(22) Filed: Jul. 26, 2021

Related U.S. Application Data
(60) Provisional application No. 63/056,644, filed on Jul. 26, 2020.

(51) Int. Cl. C07D 493/18; A61K 9/00

(52) U.S. Cl. CPC C07D 493/18; A61K 9/0014

(57) ABSTRACT

Disclosed are topical compositions and methods of treatment of skin disorders selected from Molluscum contagiosum, verrucae and warts by topical administration to a subject in need thereof of a composition comprising between about 0.5%-1.0% w/w high-purity cantharidin. Also disclosed are processes for preparation of high-purity cantharidin.

### FIELD OF THE INVENTION

The present disclosure, in some embodiments thereof, relates to topical compositions and methods of treatment of a skin disorder selected from Molluscum contagiosum, verrucae and warts by topical administration to a subject in need thereof a composition comprising between about 0.5%-1.0% w/w high purity cantharidin. Also provided are processes for preparation of high-purity cantharidin.

### BACKGROUND

Cantharidin (I) is a natural product, isolated from blister beetles insects (Meloidae) from species of the genera Mylabris and Lytta, especially Lytta vesicatoria, known as “Spanish fly”.

Cantharidin (I) is a poisonous vesicant, which has been used in compounded compositions for wart removal.

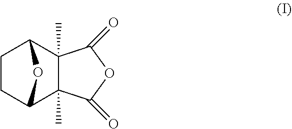

It was also produced synthetically, with the first synthesis (Stork, 1951, J. Am. Chem. Soc., 73, 4501-4501) including 11 steps, later improved (Schenck, Wirtz, R., 1953, Natunvissenshaften, 40, 531) to six steps. Dauben (J. Am. Chem. Soc. 1976, 98, 1992-1993., and J. Am. Chem. Soc. 1980, 102, 6893-6894) developed a high-pressure Diels-Alder cycloaddition synthesis, later improved by Grieco et al. (J. Am. Chem. Soc. 1990, 112, 11, 4595-4596).

There is an unmet need for compositions and prescription drugs (Rx drugs) comprising high purity cantharidin and methods of topical treatment of skin disorders like Molluscum contagiosum using those Rx drugs.

### SUMMARY OF THE INVENTION

The present disclosure provides topical compositions and methods of treatment of skin disorders selected from Molluscum contagiosum, verrucae and warts by topical administration to a subject in need thereof a composition comprising between about 0.5%-1.0% w/w high-purity cantharidin. Also disclosed are processes for preparation of high-purity cantharidin.

### DETAILED DESCRIPTION OF THE INVENTION

This application provides processes for the preparation of high-purity cantharidin, compositions comprising high-purity cantharidin and methods of treatment of skin disorders selected from Molluscum contagiosum, verrucae and warts by topical administration to a subject in need thereof of a composition comprising between about 0.5%-1.0% w/w high-purity cantharidin.

Compounded cantharidin compositions are marketed in the US.

There is presently no FDA-approved US-marketed cantharidin prescription drug (Rx drug) for topical use.

A future cantharidin Rx product will require a high-purity cantharidin active agent with an FDA-approved Drug Master File, which is presently not commercially available.

### Cantharidin Synthesis

The most recent improvement of the Diels-Alder cantharidin process was provided by Chunbin Tan, Xiaoling Liu and Hongfei Du, Rev. Roum. Chim., 2019, 64(3), 271-276, which detailed a Diels-Alder process including the cycloaddition reaction of furan with 2,5-dihydrothiophene-3,4-dicarboxylic acid anhydride (II) in an ionic liquid at atmospheric pressure,

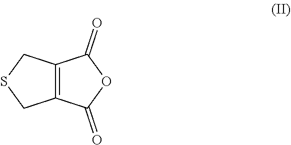

to afford the exo cycloaddition adduct 8,13-dioxa-11-thiatetracyclo[4.3.3.12,5.01,6]tridec-3-ene-7,9-dione (III) and inter alia some if its endo isomer as a side-product.

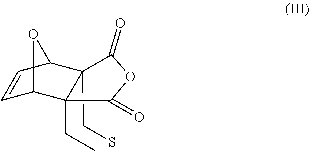

Two hydrogenation steps (reduction and desulfurization) from the (III) cantharidin intermediate afford crude cantharidin (I), which may be further purified.

### Processes for Preparation of High Purity Cantharidin

It occurred to the present inventor that cantharidin (I) and/or cantharidin intermediate (III), both being dicarboxylic anhydrides, enable a chemical purification sequence of reactions for reduction or elimination of the cantharidin (or cantharidin intermediate) impurities via the separation of the respective dicarboxylic acid metal salts.

Thus, for example, the following purification process steps of cantharidin include:
(i) conversion of cantharidin (an anhydride) to its dicarboxylic acid (a cantharidate) salt selected from Na, K, Ca, Ba, Mg and Mn cantharidate. As an example, the preparation of the disodium cantharidate salt (IV, also known as disodium cantharidin) is detailed in patent application CN102146086B.

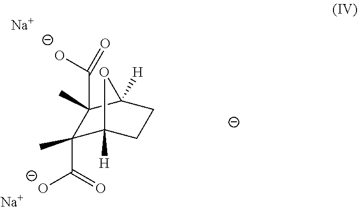

(ii) separation of the cantharidate salt of step (i) by filtration, washing with an organic solvent, crystallizing from a suitable solvent and drying.
(iii) conversion of the cantharidate salt of step (ii) to cantharidin. As an example, the catalytic process using MgCl₂ and dialkyl carbonates (Robert C. et al., ACS Catal. 2014, 4, 10, 3586-3589, Sep. 9, 2014) may be used.
(iv) crystallization of the cantharidin of step (iii) from an organic solvent selected from ethyl acetate and acetone to afford high-purity cantharidin, wherein said high-purity cantharidin has an exo-endo ratio from 85:15 to 99:1, an assay of 97%-99% w/w and a cantharinic acid content equal or lower than 0.5% w/w.

Alternatively, a similar purification process may be used starting with the cantharidine intermediate (III) via its dicarboxylic acid salt.

### Molluscum Contagiosum

Molluscum contagiosum is an infection caused by a poxvirus (molluscum contagiosum virus).

The lesions, known as Mollusca, are small, raised, and usually white, pink, or flesh-colored with a dimple or pit in the center (Centers for Disease Control and Prevention (CDC), https://www.cdc.gov/poxvirus/molluscum-contagiosum/index. html).

### Cantharidin Compositions

The present invention provides a stable topical composition for treatment of skin disorders selected from Molluscum contagiosum, verrucae and warts comprising between about 0.5%-1.0% w/w high purity cantharidin, preferably 0.7% w/w, wherein said high-purity cantharidin has an exo-endo ratio from 85:15 to 99:1, an assay of 97%-99% w/w and a cantharinic acid content equal or lower than 0.5% w/w and a carrier suitable for topical administration. The preferred cantharidin content of the composition is 0.7% w/w high purity cantharidin.

### Methods of Treatment

It is an object of the present disclosure to provide a method of treatment, prevention or alleviation of skin disorders selected from Molluscum contagiosum, verrucae and warts in a subject in need thereof, by topical administration to a subject in need thereof a therapeutically effective amount of the composition comprising between about 0.5%-1.0% w/w high purity cantharidin, wherein said high-purity cantharidin has an exo-endo ratio from 85:15 to 99:1, an assay of 97%-99% w/w and a cantharinic acid content equal or lower than 0.5% w/w and a carrier suitable for topical administration . The preferred content of the composition is 0.7% w/w high purity cantharidin.

### Embodiments

In some embodiments, there is provided a process for preparing high-purity cantharidin comprising the following steps (i)-(iv):
(i) conversion of cantharidin to a cantharidate salt selected from Na, K, Ca, Ba, Mg and Mn cantharidate;
(ii) separation of the cantharidate salt of step (i) by filtration, washing with an organic solvent, crystallizing from a suitable solvent and drying;
(iii) conversion of the cantharidate salt of step (ii) to cantharidin; and
(iv) crystallization of the cantharidin of step (iii) from an organic solvent selected from ethyl acetate and acetone to afford high-purity cantharidin, wherein said high-purity cantharidin has an exo-endo ratio from 85:15 to 99:1, an assay of 97%-99% w/w and a cantharinic acid content equal or lower than 0.5% w/w.

In some embodiments, there is provided a stable topical composition for treatment of skin disorders selected from Molluscum contagiosum, verrucae and warts comprising between about 0.5%-1.0% w/w high purity cantharidin, wherein said high-purity cantharidin has an exo-endo ratio from 85:15 to 99:1, an assay of 97%-99% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio from 85:15 to 99:1, an assay of 97%-99% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 95:5, an assay of 97%-99% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay of 97%-99% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay equal or higher than 98% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay equal or higher than 99% w/w and a cantharinic acid content equal or lower than 0.5% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay equal or higher than 99% w/w and a cantharinic acid content equal or lower than 0.1% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay equal or higher than 99% w/w and a cantharinic acid content equal or lower than 0.25% w/w, and a carrier suitable for topical administration.

In some embodiments, there is provided a stable topical composition for treatment of skin disorders selected from Molluscum contagiosum, verrucae and warts comprising 0.7% w/w high purity cantharidin, wherein said high-purity cantharidin has an exo-endo ratio equal or higher than 98:2, an assay equal or higher than 98% w/w and a cantharinic acid content equal or lower than 0.5% w/w, wherein said composition is a solution, a suspension, a cream, an ointment, a gel, a lotion, a spray, a shampoo, a patch or a foam.

In some embodiments, there is provided a method of treatment, prevention or alleviation of a skin disorder selected from Molluscum contagiosum, verrucae and warts in a subject in need thereof, by topical administration to a subject in need thereof of a therapeutically effective amount of a stable topical composition for treatment of skin disorders selected from Molluscum contagiosum, verrucae and warts comprising 0.7% w/w high purity cantharidin, wherein said high-purity cantharidin has an exo-endo ratio from 85:15 to 99:1, an assay of 97%-99% w/w and a cantharinic acid content equal or lower than 0.5% w/w, and a carrier suitable for topical administration. Definitions

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in microcapsules or formulations according to the present disclosure.

The term “about” as used herein means within an acceptable error range for a particular value as determined by one of ordinary skill in the art, which will depend in part on how the value is measured or determined, i.e., the limitations of the measurement system. For example, “about” can mean a range of between about 0.5%-1.0% w/w, more preferably 0.7%w/w of a given value. Where particular values are described in the application and claims, unless otherwise stated, the meaning of the term “about” is within an acceptable error range for the particular value or of ±10%.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

What is claimed is:

1. A process for the preparation of high-purity cantharidin comprising the following steps (i)-(iv):
(i) conversion of cantharidin to a cantharidate salt selected from Na, K, Ca, Ba, Mg and Mn cantharidate;
(ii) separation of the cantharidate salt of step (i) by filtration, washing with an organic solvent, crystallizing from a suitable solvent and drying;
(iii) conversion of the cantharidate salt of step (ii) to cantharidin; and
(iv) crystallization of the cantharidin of step (iii) from an organic solvent selected from ethyl acetate and acetone to afford high-purity cantharidin, wherein said high-purity cantharidin has an exo-endo ratio from 85:15 to 99:1, an assay of 97%-99% w/w and a cantharinic acid content equal or lower than 0.5% ww.

2. A stable topical composition for treatment of skin disorders selected from Molluscum contagiosum, verrucae and warts comprising between about 0.5%-1.0% w/w high purity cantharidin, wherein said high-purity cantharidin has an exo-endo ratio from 85:15 to 99:1, an assay of 97%-99% w/w and a cantharinic acid content equal or lower than 0.5% w/w, and a carrier suitable for topical administration.

3. The composition of claim 2, wherein said composition comprises 0.7% w/w high purity cantharidin.

4. The composition of claim 3, wherein the exo-endo ratio is equal or higher than 95:5.

5. The composition of claim 3, wherein the exo-endo ratio is equal or higher than 98:2.

6. The composition of claim 3, wherein the cantharidin assay is equal or higher than 98% w/w.

7. The composition of claim 3, wherein the cantharidin assay is equal or higher than 99% w/w.

8. The composition of claim 3, wherein the cantharinic acid content is equal or lower than 0.1% w/w.

9. The composition of claim 3, wherein the cantharinic acid content is equal or lower than 0.25% w/w.

10. The composition of claim 2, wherein said composition is a solution, a suspension, a cream, an ointment, a gel, a lotion, a spray, a shampoo, a patch or a foam.

11. A method of treatment, prevention or alleviation of a skin disorder selected from Molluscum contagiosum, verrucae and warts in a subject in need thereof, by topical administration to a subject in need thereof of a therapeutically effective amount of the composition of claim 2.
